(19) United States
(12) Patent Application Publication
(10) Pub. No.: US 2023/0332129 A1
(43) Pub. Date: Oct. 19, 2023

(54) Temperature optimized L-arabinose isomerase mutants

(71) Applicant: Clariant Produkte (Deutschland) GmbH, Frankturt am Main (DE)

(72) Inventors: Alexander FARWICK, München (DE); Jörg CLAREN, Stockdorf (DE); Heiko DIETZ, Wurster Nordseeküste (DE); Helge JOCHENS, Baierbrunn (DE); Magdalena MERTEL, Schwabhausen (DE)

(21) Appl. No.: 17/429,162

(22) PCT Filed: Mar. 16, 2020
(86) PCT No.: PCT/EP2020/057085

§ 371 Date: Aug. 6, 2021

(30) Foreign Application Priority Data
Mar. 29, 2019 (EP) 19166333.5

(51) Int. Cl. C12P 19/24; C12N 9/90

(52) U.S. Cl. CPC C12N 9/90; C12P 19/24; C12Y 503/01004

(57) ABSTRACT

The present invention relates to a temperature optimized L-arabinose isomerase with a high catalytic activity within feedstocks comprising a high concentration of divalent metal ions, a nucleic acid sequence encoding the inventive L-arabinose isomerase, a vector comprising the nucleic acid sequence encoding the inventive L-arabinose isomerase, a composition containing the inventive L-arabinose isomerase, a yeast cell comprising the inventive L- arabinose isomerase, the use of the inventive L-arabinose isomerase, the composition or the yeast cell for the fermentation of feedstock with a high content of divalent metal ions.

### CROSS-REFERENCE TO RELATED APPLICATIONS

This application claims priority to PCT Application No. PCT/EP2020/057085, filed on 16 Mar. 2020, which claims priority to EP Application No. EP19166333.5, filed 29 Mar. 2019, each of which is incorporated by reference herein in its entirety.

### BACKGROUND

Large-scale consumption of traditional, fossil fuels (petroleum-based fuels) in recent decades has contributed to high levels of pollution. This, along with the realisation that the world stock of fossil fuels is limited has stimulated new initiatives to investigate the feasibility of alternative fuels such as biomass-derived ethanol.

Although biomass-derived ethanol may be produced by the fermentation of hexose sugars obtained from agricultural products such as cane sugar and corn starch, such feedstocks are costly and industrial scale production will also severely affect global food supply.

Newer processes therefore focus on the conversion of alternative biomass material known as “lignocellulosic feedstock” such as straw and sugar cane bagasse, the so called second-generation (2G) production of biofuels. Even though lignocellulosic feedstock is available in high quantities and low in price, the feedstock is more complex and an efficient and economical conversion to ethanol is difficult.

Within the state of the art, the conversion of lignocellulosic feedstock to ethanol is known to afford several process steps, starting from a mechanical comminution by e.g. milling, a pretreatment of the comminuted material by e.g. applying steam and pressure, an enzymatic hydrolyzation of the pretreated material by using an enzymatic composition comprising cellulose and hemicellulose degrading enzymes (e.g. produced a filamentous fungus such as Trichoderma reesei) and a subsequent fermentation of the hydrolysate to ethanol by a yeast.

In addition to a large amount of hexose sugars, hydrolysate of lignocellulosic feedstock also contains a considerable amount of pentose sugars, including L-arabinose. For an economically feasible fuel production both hexose and pentose sugars must be fermented to ethanol.

The yeast Saccharomyces cerevisiae most commonly used in such processes is robust and well adapted for ethanol production, but it is unable to convert arabinose, in particular L-arabinose. Also, no naturally-occurring organisms are known which can ferment both, hexose and pentose sugars, to ethanol with a high ethanol yield and a high ethanol productivity.

Another obstacle is the high content of impurities and coproducts within the hydrolysate which interfere the fermentation process. A high content of salts leading to a high content of divalent metal ions is of particular concern and there are a number of new methods proposed for removing or limiting these impurities and coproducts. For example purification of the hydrolysate with membranes and ion exchange technology was recently disclosed. However, these purification steps significantly add to the cost of using lignocellulosic feedstock.

In addition, yeasts and enzymes used in such fermentation processes need to show a consistently optimal performance at a temperature of at least 30° C. to enable flexible process adaptions in particular during fermentation but also to limit additional cooling of the hydrolysate before entering the fermentation process to a minimum.

There is therefore a need for newly developed enzymes capable of converting L-arabinose to sugars which can be fermented by a yeast such as Saccharomyces cerevisiae to ethanol to enable a commercially-viable ethanol production from lignocellulosic feedstocks by overcoming the above referenced obstacles known from the state of the art.

### SUMMARY

The inventors of the present invention have therefore set themselves the task to develop novel L-arabinose isomerases which allow the efficient conversion of L-arabinose to L-ribulose, which is an essential intermediate for producing further products such as ethanol.

In addition, a high tolerance of divalent metal ions content within the medium was requested. Further, the novel L-arabinose isomerase should also show an optimized performance at a wider temperature range. Finally, the L-arabinose isomerase should be efficient either applied as a separate enzyme or when cloned in the yeast cell.

The present invention relates to a temperature optimized L-arabinose isomerase with a high catalytic activity within feedstocks comprising a high concentration of divalent metal ions, a nucleic acid sequence encoding the inventive L-arabinose isomerase, a vector comprising the nucleic acid sequence encoding the inventive L-arabinose isomerase, a composition containing the inventive L-arabinose isomerase, a yeast cell comprising the inventive L-arabinose isomerase, the use of the inventive L-arabinose isomerase, the composition or the yeast cell for the fermentation of feedstock with a high content of divalent metal ions.

The inventors of the present invention have now surprisingly found that this task can be solved by temperature optimized L-arabinose isomerase mutants with a high catalytic activity within feedstocks comprising a high concentration of divalent metal ions, having a sequence identity of at least 97.0% to SEQ ID NO:2.

### BRIEF DESCRIPTION OF THE DRAWINGS

FIG. 1 shows the comparison of the enzymatic activity at 30° C. for substrates containing 1 or 5 mM MnCl₂, MgCl₂ or CaCl₂ and 400 mM L-arabinose for SEQ ID NO: 8 (state of the art L-arabinose isomerase) and SEQ ID NO: 6 (L-arabinose isomerase according to the invention).

FIG. 2 shows the comparison of the enzymatic activity at 30° C. and 50° C. for substrates containing 1 or 5 mM MgCl₂ or CaCl₂ and 400 mM L-arabinose for SEQ ID NO: 8 (state of the art L-arabinose isomerase) and SEQ ID NO: 6 (L-arabinose isomerase according to the invention).

### DETAILED DESCRIPTION

In the following, the elements of the present invention will be described in more detail. These elements are listed with specific embodiments, however, it should be understood that they may be combined in any manner and in any number to create additional embodiments.

The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

The nomenclature of amino acids, peptides, nucleotides and nucleic acids within the present application follows the suggestions of IUPAC. Generally, amino acids are named within this document according to the one letter code.

The terms “DNA” and “RNA” are well known to a person skilled in the art. While DNA contains deoxyribose, RNA contains ribose (in deoxyribose there is no hydroxyl group attached to the pentose ring in the 2′ position). The complementary base to adenine is not thymine, as it is in DNA, but rather uracil, which is an unmethylated form of thymine.

If not otherwise specified, within the present invention the term “mutated” is to be understood as “substituted”, “deleted” or “inserted”. If not otherwise specified, the term “mutation” is to be understood as “substitution”, “deletion” or “insertion”. Substitutions are classified as transitions where a purine is exchanged by a purine (A <-> G) or a pyrimidine by a pyrimidine (C <->T) or transversions where a purine is exchanged by a pyridine and vice versa (C/T <-> A/G). Insertions add one or more additional nucleotides (A, C, T or G) into an oligonucleotide. The removal of one or more nucleotides from the DNA is called deletion.

The sequence identity between two amino acid sequences is determined by global alignment using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are the standard settings (EBLOSUM62 scoring matrix, gap open penalty of 10, gap extension penalty of 0.5). The output of Needle labeled “identity” is used as the percent identity.

Within the present invention, the term “L-arabinose isomerase” is to be understood as an enzyme belonging to the class EC 5.3.1.4. The L-arabinose isomerase according to the present invention is an enzyme that catalyzes the chemical reaction from L-arabinose to L-ribulose. This enzyme belongs to the family of isomerases, specifically those intramolecular oxidoreductases interconverting aldoses and ketoses. The term “L-arabinose isomerase” and enzymes belonging to class EC 5.3.1.4 are well known to a person skilled in the art, who is able to identify and differentiate an L-arabinose isomerase from other enzymes.

Within the present invention, the term “temperature optimized L-arabinose isomerase” refers to L-arabinose isomerases which show a catalytic activity of at least 28.5 kat/mol enzyme within a temperature range of from 30° C. to 50° C. during an incubation of 20 minutes at a pH of 7 and on a substrate containing 400 mM L-arabinose and comprising 1 mM Ca²⁺ or 1 mM Mg²⁺. The term “kat” (abbreviation for katal) is the unit of catalytic activity in the International System of Units (SI). It quantifies the catalytic activity of enzymes (that is, measuring the enzymatic activity level in enzyme catalysis).

Within the present invention, the term “high catalytic activity” refers to a catalytic activity of at least 28.5 kat/mol enzyme within a temperature range of from 30° C. to 50° C. during an incubation of 20 minutes at a pH of 7 and on a substrate containing 400 mM L-arabinose and comprising 1 mM Ca²⁺ or 1 mM Mg²⁺.

Within the present invention, the term feedstock is to be understood as referring to any feedstock comprising at least 0.5 g/l L-arabinose, at least 5 g/l of D-glucose and shows a content of at least 2 mM of divalent metal ions. Exemplary suitable feedstocks comprise from 0.5 to 15 g/l L-arabinose, from 5 to 200 g/l of D-glucose and show a content of from 2 to 50 mM of divalent metal ions. Particularly suitable feedstocks comprise from 0.5 to 10 g/l L-arabinose, from 5 to 150 g/l of D-glucose, from 5 to 60 g/l D-xylose and show a total content of from 5 to 30 mM of Mg²⁺ and Ca²⁺ ions. Further particularly preferred feedstocks are hydrolysates from cellulosic and/or lignocellulosic biomass such as hydrolysate from cereal straw and/or husks, sugar cane bagasse, switch grass, rice bran, corn stover, paper, raw paper pulp, waste-paper, wood, agricultural residues, forestry residues, municipal solid wastes hull products and mixtures thereof. The production of such a hydrolysate is known to a person skilled in the art and for exampled described within EP 2015723515 or WO 2017042019 which are herein incorporated by reference.

Within the present invention, the term “high concentration of divalent metal ions” is to be understood as a concentration of divalent metal ions of at least 2 mM, at least 5 mM, at least 10 mM or at least 15 mM. A concentration of divalent metal ions of from 2 to 50 mM, from 2 to 45 mM or from 5 to 35 mM is also within the scope of the present invention. Particularly suitable feedstocks comprise Mn²⁺, Mg²⁺ and/or Ca²⁺ in a concentration of from 1 to 40 mM, of from 2 to 35 mM or from 5 to 30 mM. The term “divalent metal ions” is well known to a person skilled in the art and to be understood as comprising Ca²⁺, Mn²⁺, Mg²⁺, Ba²⁺, Cu^{Z+}, Zn²⁺.

The temperature optimized L-arabinose isomerase according to the present invention has a sequence identity of at least 97.0 % to SEQ ID NO: 2, wherein L-arabinose isomerases with a sequence identity of at least 97.5, at least 98.0%, at least 98.5%, at least 99.0%, at least 99.5 or at least 99.9% are particularly suitable for the inventive purpose.

Further particularly advantageous temperature optimized L-arabinose isomerases according to the present invention show a sequence identity of at least 97.0% to SEQ ID NO: 2 and further comprise SEQ ID NO: 4 with at least one mutation selected from the group consisting of deletions and substitutions at position 40, 41, 42, 43, 239, 240, 241, 242, 243, 244, 246, 247, 248, 249, 250 and 251. Within particularly advantageous temperature optimized isomerase L-arabinoses this at least one mutation is a substitution selected from the group consisting of substitutions at position 40, 41, 43, 239, 246, 247, 248, 249, 250 and 251, wherein it is even more particularly advantageous to select the at least one substitution from the group consisting of A40K, S41K, K43D, M239I, H246P, D247K, K248A, Y249S, V250M and H251D. Within other particularly advantageous temperature optimized L-arabinose isomerases this at least one mutation is a deletion selected from the group consisting of deletions at position 42, 240, 241, 242, 243 and 244, wherein it is even more advantageous to select the at least one deletion from the group consisting of G42, V240, E241, G242, D243 and N244. Combinations of these specified mutations are also within the scope of the present invention.

Other particularly advantageous temperature optimized L-arabinose isomerases according to the present invention show a sequence identity of at least 97.0% to SEQ ID NO: 2 and further comprise SEQ ID NO: 4 with at least one substitution at position 40, 41 and 43 and/or at position 239, 246, 247, 248, 249, 250 and 251. Further particularly advantageous temperature optimized L-arabinose isomerases according to the present invention show a sequence identity of at least 97.0% to SEQ ID NO: 2 and further comprise SEQ ID NO: 4 with at least one deletion at position 42 and/or at position 240, 241, 242, 243 and 244. Combinations of these specified mutations are also within the scope of the present invention.

Other particularly advantageous temperature optimized L-arabinose isomerases according to the present invention are selected from the group consisting of SEQ ID NO: 6, SEQ ID NO: 8, SEQ ID NO: 11, SEQ ID NO: 13, SEQ ID NO: 15 and SEQ ID NO: 17.

Other particularly advantageous temperature optimized L-arabinose isomerases according to the present invention have a sequence identity of at least 97.0% to SEQ ID NO: 2 and a catalytic activity of at least 28.5 kat/mol enzyme within feedstocks of an L-arabinose content of 400 mM and a content of MgCl₂ of 5 mM when incubated at 30° C. for 20 minutes at pH 7, wherein L-arabinose isomerases with a catalytic activity of at least 30.0 kat/mol enzyme and a content of 5 mM CaCl₂ are also advantageous.

Within another embodiment, the present invention further provides a nucleic acid encoding the temperature optimized L-arabinose isomerase according to the present invention.

Within another embodiment, the present invention further provides vectors comprising the nucleic acid of the present invention. Examples for episomally maintained vectors are derivatives of bacterial plasmids, yeast plasmids, centromer based linear DNA, constructs of viral origin like SV40, phage DNA, fungal ARS based DNA-vehicles, baculovirus, vaccinia, adenovirus, fowl pox virus, and pseudorabies as well as vectors derived from combinations of plasmids and phage or viral DNA. A suitable expression vector according to the present invention may comprise one or more genetic elements representing promotor sequences, transcription initiation sites, elements for the initiation of translation, and functional elements for protein export that are translationally coupled to the nucleic acid according to the present invention. In addition to the inventive nucleic acid, the vector according to the present invention may encode more than one polypeptide including more than one L-arabinose isomerase or may encode a fusion polypeptide comprising the L-arabinose isomerase according to the invention. The vector according to the present invention may be episomally maintained in the host cell or integrated into the chromosome of the host.

Within a further embodiment, the present invention provides a composition comprising the temperature optimized L-arabinose isomerase according to the present invention. The composition may be a liquid or dry composition. Liquid compositions according to the present invention may only contain the inventive L-arabinose isomerase, preferably in a highly purified form. Usually, however, a stabilizer such as glycerol, sorbitol or mono propylene glycol is also contained. The liquid composition may also comprise other additives, such as salts, sugars, preservatives, pH-adjusting agents and/or proteins. Typical liquid compositions are aqueous or oil-based slurries. Dry compositions may be spray dried compositions, in which case the composition need not contain anything more than the inventive L-arabinose isomerase in a dry form. Usually, however, dry compositions are so-called granulates which may readily be mixed with the substrate/feedstock. Agglomeration granulates coated with the inventive arabinose isomerase can be prepared using agglomeration techniques in a high shear mixer. Absorption granulates can be prepared by having cores of a carrier material to absorb/be coated by inventive L-arabinose isomerase.

The carrier material may be a biocompatible non-biodegradable. Typical filler materials used in agglomeration techniques include salts, such as disodium sulphate. Other fillers are kaolin, talc, magnesium, aluminum, silicate and cellulose fibers. Optionally, binders such as dextrins are also included in agglomeration granulates.

The composition may further comprise at least one enzyme selected from the group consisting cellulases such as endoglucanases, cellobiohydrolases and beta-glucosidases, hemicellulases such as xylanases, xylosidases, mannanases and mannosidases, pectinases, ribulokinases and ribulose-5-phosphate-4-epimerases.

Within a further embodiment, the present invention provides a yeast cell comprising the temperature optimized L-arabinose isomerase according to the present invention. Within a particularly advantageous embodiment the yeast cell according to the present invention is selected from the group consisting of Saccharomyces, Kluyveromyces, Schizosaccharomyces, Candida, Yarrowia, Komagataella, Pichia and Hansenula wherein a yeast cell selected from the group consisting of Saccharomyces cerevisiae, Saccharomyces bayanus, Saccharomyces uravum, Saccharomyces pastorianus, Saccharomyces kudriavzevii, Saccharomyces mikatae, Saccharomyces carlsbergensis, Schizosaccharomyces pombe, Kluyveromyces marxianus, Yarrowina lipolytica, Hansenula polymorpha, Pichia angusta, Komagataella pastoris and Pichia pastoris is even more suitable for the inventive purpose.

The yeast cell according to the present invention may comprise one or more vectors according to the present invention. In addition or alternatively, the nucleic acid is integrated in the genome of the yeast cell.

Within a further embodiment, the present invention provides the use of the temperature optimized L-arabinose isomerase or of the inventive composition comprising the temperature optimized L-arabinose isomerase or the inventive yeast cell comprising the temperature optimized L-arabinose isomerase within a fermentation process of feedstock with a high content of divalent metal ions. Such fermentation processes are well known to a person skilled in the art.

Feedstocks comprising arabinose, particularly L-arabinose, can also serve as precursors to platform chemicals that can be converted to a wide variety of products. Continued advances in sugar production and conversion are key factors in the future of biomass-based fuels and chemicals. It is therefore particularly suitable that the temperature optimized L-arabinose isomerase, the yeast cell and/or the composition according to the present invention is used for a fermentation process for the production of a fermentation product such as e.g. ethanol, butanol, lactic acid, 3 -hydroxy-propionic acid, acrylic acid, acetic acid, succinic acid, citric acid, malic acid, fumaric acid, itaconic acid, an amino acid, 1,3-propane-diol, ethylene, glycerol, a ß-lactam antibiotic, such as Penicillin and fermentative derivatives thereof.

### GENERALLY PREFERRED EMBODIMENTS

In the following, generally preferred embodiments of the present invention are listed. These embodiments illustrate particularly advantageous embodiments of the present inventions and do not limit the scope of the present invention and claims in any respect.

### Generally Preferred Embodiment A

Temperature optimized L-arabinose isomerase with a high catalytic activity within feedstocks comprising a high concentration of divalent metal ions, having a sequence identity of at least 97.0% to SEQ ID NO:2 and comprising SEQ ID NO: 4 with a substitution at position 40, 41 and 43 and/or at position 239, 246, 247, 248, 249, 250 and 251.

### Generally Preferred Embodiment B

Temperature optimized L-arabinose isomerase according to generally preferred embodiment A with a deletion at position 42 and/or at position 240, 241, 242, 243 and 244.

### Generally Preferred Embodiment C

Temperature optimized L-arabinose isomerase according to generally preferred embodiment A or B, wherein the L-arabinose isomerases show a catalytic activity of at least 28.5 kat/mol enzyme within feedstocks of an L-arabinose content of 400 mM and a content of MgCl₂ of 5 mM when incubated at 30° C. for 20 minutes at pH 7.

### Generally Preferred Embodiment D

Temperature optimized L-arabinose isomerase according to generally preferred embodiment C, wherein the L-arabinose isomerases show a catalytic activity of at least 30.0 kat/mol enzyme within feedstocks of an L-arabinose content of 400 mM and a content of CaCl₂ of 5 mM when incubated at 30° C. for 20 minutes at pH 7.

### Generally Preferred Embodiment E

Temperature optimized L-arabinose isomerase according to generally preferred embodiment A or B, wherein the L-arabinose isomerases show a catalytic activity of at least 28.5 kat/mol enzyme within feedstocks of an L-arabinose content of 400 mM and a content of MgCl₂ of 5 mM when incubated at 30° C. for 20 minutes at pH 7.

### Generally Preferred Embodiment F

Temperature optimized L-arabinose isomerase according to generally preferred embodiment A or B, wherein the L-arabinose isomerases show a catalytic activity of at least 28.5 kat/mol enzyme within feedstocks containing from 0.5 to 15 g/l L-arabinose, from 5 to 200 g/l of D-glucose and a content of from 2 to 50 mM of divalent metal ions when incubated at 30° C. for 20 minutes at pH 7.

### Generally Preferred Embodiment G

Temperature optimized L-arabinose isomerase according to generally preferred embodiment A or B, wherein the L-arabinose isomerases show a catalytic activity of at least 28.5 kat/mol enzyme within feedstocks containing from 0.5 to 10 g/l L-arabinose, from 5 to 100 g/l of D-glucose, from 5 to 50 g/l D-xylose and a total content of from 5 to 30 mM of Mg²⁺ and Ca²⁺ ions when incubated at 30° C. for 20 minutes at pH 7 and wherein the feedstock is a hydrolysate of lignocellulose containing biomass such as cereal straw, bagasse, cornstover, rice bran, rice hulls, rice straw or mixtures thereof.

### Generally Preferred Embodiment H

Temperature optimized L-arabinose isomerase according to any of generally preferred embodiment A to G, having a sequence identity of at least 98.5% to SEQ ID NO:2 and comprising SEQ ID NO: 4 with a substitution at position 40, 41 and 43 and/or at position 239, 246, 247, 248, 249, 250 and 251.

### Generally Preferred Embodiment I

Temperature optimized L-arabinose isomerase according to any of generally preferred embodiment A to G, having a sequence identity of at least 99.5% to SEQ ID NO:2 and comprising SEQ ID NO: 4 with at least one deletion at position 42 and/or at position 240, 241, 242, 243 and 244.

### Generally Preferred Embodiment J

Nucleic acid sequence encoding a temperature optimized L-arabinose isomerase according to any of generally preferred embodiments A to G.

### Generally Preferred Embodiment K

Composition containing a temperature optimized L-arabinose isomerase according to any of generally preferred embodiments A to G.

### Generally Preferred Embodiment L

Yeast cell comprising a temperature optimized L-arabinose isomerase according to any of generally preferred embodiments A to G.

### Generally Preferred Embodiment M

Use of a temperature optimized L-arabinose isomerase according to any of generally preferred embodiments A to G, a composition according to generally preferred embodiment I or a yeast cell according to generally preferred embodiment J within a fermentation process of feedstocks containing from 0.5 to 10 g/l L-arabinose, from 5 to 100 g/l of D-glucose, from 5 to 50 g/l D-xylose and a total content of from 5 to 30 mM of Mg²⁺ and Ca²⁺ ions and wherein the feedstock is a hydrolysate of lignocellulose containing biomass such as cereal straw, bagasse, cornstover, rice bran, rice hulls, rice straw or mixtures thereof.

### Generally Preferred Embodiment N

Use according to generally preferred embodiment M, wherein the fermentation process is a fermentation process for the production of a fermentation product such as e.g. ethanol, butanol, lactic acid, 3 -hydroxy-propionic acid, acrylic acid, acetic acid, succinic acid, citric acid, malic acid, fumaric acid, itaconic acid, an amino acid, 1,3-propane-diol, ethylene, glycerol, a ß-lactam antibiotic, such as Penicillin and fermentative derivatives thereof.

### EXAMPLES

In the following the present invention is described by the examples. The examples are considered for illustrative purpose only and do not limit the scope of the present invention and claims in any respect.

### Example 1

### Cloning of Vectors for Expression of L-Arabinose Isomerase Genes in Escherichia Coli

Methods for manipulation of nucleic acid molecules are generally known to the skilled person in the field and are here introduced by reference (1. Molecular cloning: a laboratory manual, Michael R. Green and Joseph Sambrook, 4th edition; 2. Current Protocols in Molecular Biology, ISSN: 1934-3639).

SEQ ID NO: 1 and 3, coding for SEQ ID NO: 2 and 4, respectively, were synthesized by Geneart (Regensburg, Germany) and then used as template in PCR reactions for cloning. A sequence coding for a N-terminal 6xHis-tag followed by a TEV protease site (ENLYFQS) was added to the ORF in two successive PCRs. The resulting open reading frames are SEQ ID NO: 5 and SEQ ID NO: 7, respectively. The corresponding amino acid sequences are SEQ ID NO: 6 and SEQ ID NO: 8. For the first PCR reaction, primer pairs were designed to match the 5′ and 3′ ends of SEQ ID NO: 1 and 3, respectively. The forward primer (binding to the 5′ end of the respective ORF) has a 5′ extension containing the sequence of the TEV protease site and a part of the 6xHis-tag sequence (12 bp). The reverse primer has a 5′ extension suitable for Gibson cloning into the target vector. The forward primer for the second reaction was designed to bind the 5′ extension of the first forward primer and has a 5′extension containing the residual 6xHis-tag sequence and a sequence allowing Gibson cloning into the target vector. The reverse primer for the first and second reaction are identical. Both PCR reactions were set up using Q5 High Fidelity DNA Polymerase (HF-Buffer system) following the recommendations of the supplier for dNTP, primer and buffer concentrations. The amplification of the PCR products was done in an Eppendorf Thermocycler using the standard program for Phusion Polymerase (98° C., 30 sec initial denaturation followed by 30 cycles of 98° C., 20 sec - 60° C., 20 sec - 72° C., 30 sec and a final elongation phase at 72° C. for 5 minutes. The PCR products of expected size were purified by preparative, ethidium bromide stained TAE-Agarose gel electrophoresis and recovered from the Gel using the Promega Wizard SV-PCR and Gel Purification Kit.

Plasmid pET-22b was linearized by digestion with restriction endonucleases HindIII and Ndel and the digested fragment separated by agarose gel electrophoresis. The linearized vector-backbone was recovered from the gel following the instructions of the Promega Wizard SV-PCR and Gel Purification Kit. The amplified PCR product was cloned into the digested vector-backbone using a Gibson cloning methods (NEBuilder) according to manufacturer’s indications (vector-insert ration 1:3, 0.1 pmol total DNA, 20 µl reaction). Transformation was performed into competent E. coli Mach1 cells according to the supplier’s protocol. Transformants were grown over night on LB-Ampicillin plates and tested for correct plasmid by plasmid mini-prep and control digestion as well as DNA sequencing. A larger quantity of plasmid DNA was prepared from a confirmed clone using the Promega PureYield™ Plasmid Midiprep System.

### Expression of L-Arabinose Isomerase in E. Coli and L-Arabinose Isomerase Purification

The L-arabinose isomerases were expressed in E. coli Rosetta™ cells under IPTG (Isopropyl β-D-1-thiogalactopyranoside) inducible promoter by using standard molecular biology technics. A preculture of 200 ml Lysogeny Broth containing 34 µg/mL Chloramphenicol and 100 µg/ml Ampicillin was inoculated directly with the transformed E. coli cells and grown over-night at 37° C. and 250 rpm shaking. Eight main cultures of 250 ml Terrific Broth with 100 µg/ml ampicillin in 2 L shake flasks were inoculated to an OD₆₀₀ of 0.1 with the over-night culture and incubated at 37° C. and 250 rpm shaking. When the cultures reached an OD₆₀₀ of 1.0, the temperature was reduced to 20° C. and the expression of the Al induced by addition of 1 mM IPTG. After 21.5 hours cells were harvested by centrifugation (10 min, 10,000 × g). The pellets were washed once with saline (0.9% NaCl) and combined into one tube. The pellet was then stored at -20° C. until cell lysis.

For cell lysis, the cell pellet was thawed, resuspended in 70 ml binding buffer (20 mM Trizma base, 500 mM NaCl, 20 mM Imidazole, pH 7.4) and protease inhibitor (complete™, EDTA-free Protease Inhibitor Cocktail), Lysozyme and Benzonase Nuclease (Sigma-Aldrich, 62970 and E1014) were added. The cell suspension was incubated on ice for 30 minutes. The cells were then lysed by ultrasonification while being kept on ice with the following settings: 4 × 1 min, amplitude 70%, cycle 0.6, with 1 min breaks in between. The lysed cells were centrifuged for 60 min at 20,000 × g at 4° C. The cleared supernatant was filtered (0.2 µm) and used for purification of the respective Al.

Purification was done using an Äkta explorer 900 and a 5 mL HisTrap HP column (GE life sciences). The column was equilibrated with binding buffer and the sample then loaded onto the column. The column was washed with binding buffer until the absorption at 280 nm had normalized. Elution was done with a gradient over 10 column volumes (50 mL, flow 3 ml/min, 17 minutes) with elution buffer (20 mM Trizma base, 500 mM NaCl, 500 mM Imidazole, pH 7.4). Fractions of 2 ml were collected and later analyzed by SDS-PAGE (Biorad Criterion XT 4-12% Bis-Tris) according manufacturer’s instructions. Fractions containing the respective Al protein were pooled and, to remove divalent cations from the protein, were dialyzed at 4° C. against 2× 2 L of dialysis buffer (50 mM HEPES, pH 7.0, 10 mM EDTA) over 96 h with one buffer change. Al concentration was determined photometrically via absorption at 280 nm.

Proteins with SEQ ID NO: 6 and SEQ ID NO: 8 were obtained at concentrations of 15.4 and 12.3 g/L, respectively.

### Measurement of L-Arabinose Isomerase Activity

Measurements to determine the catalytic activity of the Al proteins were conducted with the purified proteins in an end-point measurement. The amount of L-ribulose, product of Al activity with L-arabinose as a substrate, was quantified by HPLC (Dionex Ulimate 3000, with RI detector Shodex RI-101) using a Biorad Aminex HPX-87P column (with respective precolumn). The settings are the following:
eluent: MilliQ water
flow rate: 0.6 ml/min
column oven 80° C.
run time: 40 min
injection volume 20 µl
detection: RI, 50° C., Polarity (plus), Data Collection Rate 10 Hz

The calibration range for L-arabinose and L-ribulose are 0.25 - 10 mg/ml and 0.05 - 2 mg/ml, respectively.

Tests were conducted beforehand to establish the reaction conditions, esp. the amount of protein and reaction time to ensure a measurement in the linear range. The final composition of the reactions is shown in table 1.

| | |
|---|---|
| Component | Final concentration |
| HEPES pH 7 | 100 mM |
| MnCl₂ / MgCl₂ / CaCl₂ | ⅕ mM |
| L-arabinose | 400 mM |
| purified Al protein | 0.375 µM |

100 µl of a protein solution (purified Al diluted to 0.75 µM in 100 mM HEPES, pH 7) were added to 20 µl of a MnCl₂ / MgCl₂ / CaCl₂ solution (10 or 50 mM in 100 mM HEPES, pH 7). 80 µl of a concentrated L-arabinose solution (1 M in 100 mM HEPES, pH 7) were added and the reaction was mixed and centrifuged. The reactions were performed at 30° C. or 50° C. for 20 minutes in a thermocycler. The reactions were stopped by heat treatment at 95° C. for 10 min, then diluted 1:5 with water, filtered and analyzed by HPLC. Each reaction was done in triplicate. The enzyme activity was calculated from the amount of L-ribulose (in mole) produced after 1200 seconds by 75 picomoles of enzyme. The unit is kat/mol_{enzyme} with kat being mol_{ribulose}/s.

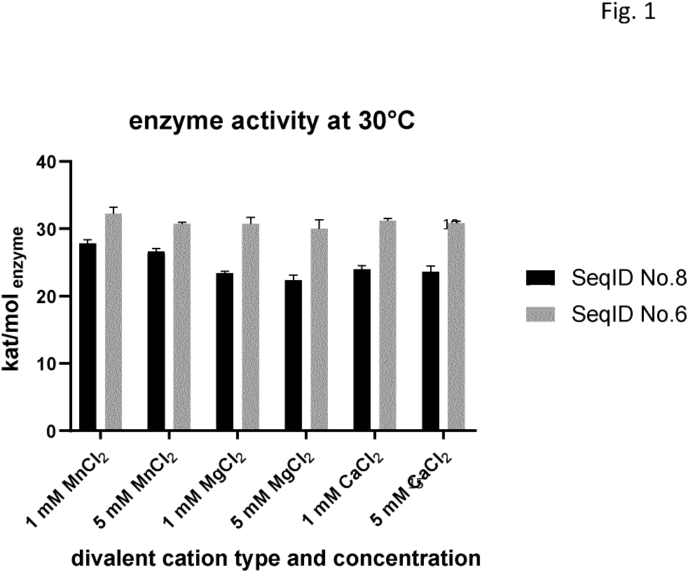

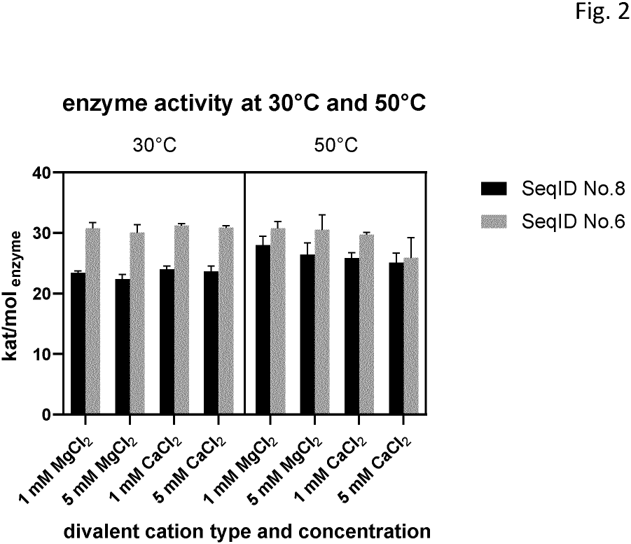

In FIG. 1 it can be seen, that at 30° C. the enzyme according to SEQ ID NO: 6 has a higher catalytic activity than the enzyme according to SEQ ID NO: 8 at low and high concentrations of all divalent metal cations. The catalytic activity is at least 15% higher in the presence of Mn²⁺ and at least 30% higher in the presence of Mg²⁺ and Ca²⁺. FIG. 2 shows furthermore that the catalytic activity of the enzyme according to SEQ ID NO: 6 is superior to the enzyme according to SEQ ID NO: 8 for divalent cations Mg²⁺ and Ca²⁺ at 30° C. and an elevated temperature of 50° C. Taken together, this shows that the L-arabinose isomerase according to SEQ ID NO: 6 shows a significantly increased temperature tolerance compare to the enzyme according to SEQ ID NO: 8 with a high catalytic activity in the presence of high concentrations of divalent metal cations.

### SEQUENCE LISTING DESCRIPTION

SEQ ID NO: 1: DNA sequence of modified L-arabinose isomerase, codon-optimized
SEQ ID NO: 2: amino acid sequence of modified L-arabinose isomerase
SEQ ID NO: 3: DNA sequence of wildtype L-arabinose isomerase, codon-optimized
SEQ ID NO: 4: amino acid sequence of wildtype L-arabinose isomerase from Lactobacillus antri DSM 16041
SEQ ID NO: 5: DNA sequence of modified L-arabinose isomerase, N-terminal 6xhis-tag, codon-optimized
SEQ ID NO: 6: amino acid sequence of modified L-arabinose isomerase, N-terminal 6xhis-tag
SEQ ID NO: 7: DNA sequence of wildtype L-arabinose isomerase, N-terminal 6xhis-tag, codon-optimized
SEQ ID NO: 8: amino acid sequence of wildtype L-arabinose isomerase, N-terminal 6xhis-tag
SEQ ID NO: 9: DNA sequence of modified L-arabinose isomerase, codon-optimized
SEQ ID NO: 10: DNA sequence of modified L-arabinose isomerase, codon-optimized
SEQ ID NO: 11: amino acid sequence of modified L-arabinose isomerase
SEQ ID NO: 12: DNA sequence of modified L-arabinose isomerase, codon-optimized
SEQ ID NO: 13: amino acid sequence of modified L-arabinose isomerase
SEQ ID NO: 14: DNA sequence of modified L-arabinose isomerase, N-terminal 6xhis-tag, codon-optimized
SEQ ID NO: 15: amino acid sequence of modified L-arabinose isomerase, N-terminal 6xhis-tag
SEQ ID NO: 16: DNA sequence of modified L-arabinose isomerase, N-terminal 6xhis-tag, codon-optimized
SEQ ID NO: 17: amino acid sequence of modified L-arabinose isomerase, N-terminal 6xhis-tag

1. Temperature optimized L-arabinose isomerase with a high catalytic activity within feedstocks comprising a high concentration of divalent metal ions, having a sequence identity of at least 97.0% to SEQ ID NO:2.

2. Temperature optimized L-arabinose isomerase according to claim 1 comprising SEQ ID NO: 4 with at least one mutation selected from the group consisting of deletions and mutations at position 40, 41, 42, 43, 239, 240, 241, 242, 243, 244, 246, 247, 248, 249, 250 and 251.

3. Temperature optimized L-arabinose isomerase according to claim 2, wherein the at least one mutation is a substitution selected from the group consisting of substitutions at position 40, 41, 43, 239, 246, 247, 248, 249, 250 and 251.

4. Temperature optimized L-arabinose isomerase according to claim 2, wherein the at least one mutation is a substitution selected from the group consisting of A40K, S41K, K43D, M2391, H246P, D247K, K248A, Y249S, V250M and H251D.

5. Temperature optimized L-arabinose isomerase according to claim 2, wherein the at least one mutation is a deletion selected from the group consisting of deletions at position 42, 240, 241, 242, 243 and 244.

6. Temperature optimized L-arabinose isomerase according to claim 5, wherein the at least one mutation is a deletion selected from the group consisting of G42, V240, E241, G242, D243 and N244.

7. Temperature optimized L-arabinose isomerase according to claim 2, comprising SEQ ID NO: 4 with a substitution at position 40, 41 and 43 and/or at position 239, 246, 247, 248, 249, 250 and 251.

8. Temperature optimized L-arabinose isomerase according to claim 2, comprising SEQ ID NO: 4 with a deletion at position 42 and/or at position 240, 241, 242, 243 and 244.

9. Temperature optimized L-arabinose isomerase according to claim 1, having a sequence identity of at least 98.0% to SEQ ID NO: 2.

10. Temperature optimized L-arabinose isomerase according to claim 1, having a sequence identity of at least 99.0% to SEQ ID NO: 2.

11. Temperature optimized L-arabinose isomerase according to claim 1 selected from the group consisting of SEQ ID NO: 6, SEQ ID NO: 11, SEQ ID NO: 13, SEQ ID NO: 15 and SEQ ID NO 17.

12. Temperature optimized L-arabinose isomerase according to claim 1, having a catalytic activity of at least 28.5 kat/mol enzyme within feedstocks of an L-arabinose content of 400 mM and a content of MgCl2 of 5 mM when incubated at 30° C. for 20 minutes at pH 7.

13. Nucleic acid sequence encoding a temperature optimized L-arabinose isomerase according to claim 1.

14. Use of the temperature optimized L-arabinose isomerase according to claim 1 within a fermentation process of lignocellulosic hydrolysate.

15. Composition containing the temperature optimized L-arabinose isomerase according to claim 1.

16. Use of the composition according to claim 15 within a fermentation process of lignocellulosic hydrolysate.

17. Vector comprising the nucleic acid sequence according to claim 13.

18. Yeast cell comprising the nucleic acid sequence according to claim 13.

19. Use of the yeast cell according to claim 18 within a fermentation process of feedstock with a high content of divalent metal ions.

20. Temperature optimized L-arabinose isomerase according to claim 7, comprising SEQ ID NO: 4 with a deletion at position 42 and/or at position 240, 241, 242, 243 and 244.


SEQUENCE LISTING (17 sequences)

SEQ ID NO: 1 - DNA, 1407 bp, Artificial Sequence
  modified L-arabinose isomerase, codon-optimized
atgttgaagaccccagattacgaattctggttcgctgtcggttcccaaccattgtacggtccagaagctttggaacaagtcgaaaaggatggtagaaagttggtcgatggtttgaacaagaaggacttgccatacaaggtcgttttcaagttggtcgctaccactgctgattccatcaccaagttcatgaaggatgctaactacaacgataaggtcgctggtgtcatgacctggatgcataccttctccccagctaagaactggatcagaggtacccaattgttacaaaagccattgttacatttggctacccaaatgttggatcatatcccattcgataccatcgacttcgattacatgaacttgaaccaatccgctcatggtgatagagaatacgattacatcaacgctagattgggtgtcccagaaaagatcgtctacggttggtggaacgatccagaagtccaagaagaaatcgctgattggcaaaaggtcgctatcgcctacgatatgtccttcaagatcaagattgctagattcggtgataccatgagagatgtcgctgttaccgaaggtgataaggtcgctgcccaaatcaagttgggttggaccgtcgattactggccagtcgctgaattggtcgctgccgtcaacgctgtctccgaagctgaaatcgatgaacaatacaagagattggaagataactacgacattgacccaaaggcttctatggattccgtcagataccaattgagagaatacttgggtatcaagcatttcttggatgaaaagggttacgatgctttcaccactaacttccaagatttggaaggtttggaacaattgccaggtttggctgtccaattgttaatgatcgatggttacggtttcggtgctgaaggtgatttcaagtccgctggtttgtctagattgttaaagatcgctgccgaaaacaaggaaaccgctttcatggaagattacaccttggatttgagagctggtcatcaagctatcatgggttcccatatgttggaagtcgatccaaccttggcttccgataagccaagagtcgaagttcatccattggatatcggtgacaaggctgatccagctagattggtcttcaccggttccgaaggtgaaggtatcgatgtcaccttgtcctacttcgatgacggttacaagttcatcgcttacgatgtcgactgtgctaagccagaaaaggaaatgccaaacttgccagtcgctaagcaaatgtggaccccaaagtgtggtttgaagaaaggtgctaccgcctggatgcataacggtggtggtcatcacaccgtcttgtccatgaacttgaccatggatcaaatggctaccttggctaacttgttcggtatcgaattgattgatatcaagtga

SEQ ID NO: 2 - protein, 468 aa, Artificial Sequence
  modified L-arabinose isomerase
MLKTPDYEFWFAVGSQPLYGPEALEQVEKDGRKLVDGLNKKDLPYKVVFKLVATTADSITKFMKDANYNDKVAGVMTWMHTFSPAKNWIRGTQLLQKPLLHLATQMLDHIPFDTIDFDYMNLNQSAHGDREYDYINARLGVPEKIVYGWWNDPEVQEEIADWQKVAIAYDMSFKIKIARFGDTMRDVAVTEGDKVAAQIKLGWTVDYWPVAELVAAVNAVSEAEIDEQYKRLEDNYDIDPKASMDSVRYQLREYLGIKHFLDEKGYDAFTTNFQDLEGLEQLPGLAVQLLMIDGYGFGAEGDFKSAGLSRLLKIAAENKETAFMEDYTLDLRAGHQAIMGSHMLEVDPTLASDKPRVEVHPLDIGDKADPARLVFTGSEGEGIDVTLSYFDDGYKFIAYDVDCAKPEKEMPNLPVAKQMWTPKCGLKKGATAWMHNGGGHHTVLSMNLTMDQMATLANLFGIELIDIK

SEQ ID NO: 3 - DNA, 1425 bp, Artificial Sequence
  wildtype L-arabinose isomerase, codon-optimized
atgttgaagaccccagattacgaattctggttcgctgtcggttcccaaccattgtacggtccagaagctttggaacaagtcgaaaaggatggtagaaagttggtcgatggtttgaacgcttccggtaagttgccatacaaggtcgttttcaagttggtcgctaccactgctgattccatcaccaagttcatgaaggatgctaactacaacgataaggtcgctggtgtcatgacctggatgcataccttctccccagctaagaactggatcagaggtacccaattgttacaaaagccattgttacatttggctacccaaatgttggatcatatcccattcgataccatcgacttcgattacatgaacttgaaccaatccgctcatggtgatagagaatacgattacatcaacgctagattgggtgtcccagaaaagatcgtctacggttggtggaacgatccagaagtccaagaagaaatcgctgattggcaaaaggtcgctatcgcctacgatatgtccttcaagatcaagattgctagattcggtgataccatgagagatgtcgctgttaccgaaggtgataaggtcgctgcccaaatcaagttgggttggaccgtcgattactggccagtcgctgaattggtcgctgccgtcaacgctgtctccgaagctgaaatcgatgaacaatacaagagattggaagataactacgacatggtcgaaggtgataacgaccatgataagtacgtccattccgtcagataccaattgagagaatacttgggtatcaagcatttcttggatgaaaagggttacgatgctttcaccactaacttccaagatttggaaggtttggaacaattgccaggtttggctgtccaattgttaatgatcgatggttacggtttcggtgctgaaggtgatttcaagtccgctggtttgtctagattgttaaagatcgctgccgaaaacaaggaaaccgctttcatggaagattacaccttggatttgagagctggtcatcaagctatcatgggttcccatatgttggaagtcgatccaaccttggcttccgataagccaagagtcgaagttcatccattggatatcggtgacaaggctgatccagctagattggtcttcaccggttccgaaggtgaaggtatcgatgtcaccttgtcctacttcgatgacggttacaagttcatcgcttacgatgtcgactgtgctaagccagaaaaggaaatgccaaacttgccagtcgctaagcaaatgtggaccccaaagtgtggtttgaagaaaggtgctaccgcctggatgcataacggtggtggtcatcacaccgtcttgtccatgaacttgaccatggatcaaatggctaccttggctaacttgttcggtatcgaattgattgatatcaagtga

SEQ ID NO: 4 - protein, 474 aa, Lactobacillus antri DSM 16041
  wildtype L-arabinose isomerase
MLKTPDYEFWFAVGSQPLYGPEALEQVEKDGRKLVDGLNASGKLPYKVVFKLVATTADSITKFMKDANYNDKVAGVMTWMHTFSPAKNWIRGTQLLQKPLLHLATQMLDHIPFDTIDFDYMNLNQSAHGDREYDYINARLGVPEKIVYGWWNDPEVQEEIADWQKVAIAYDMSFKIKIARFGDTMRDVAVTEGDKVAAQIKLGWTVDYWPVAELVAAVNAVSEAEIDEQYKRLEDNYDMVEGDNDHDKYVHSVRYQLREYLGIKHFLDEKGYDAFTTNFQDLEGLEQLPGLAVQLLMIDGYGFGAEGDFKSAGLSRLLKIAAENKETAFMEDYTLDLRAGHQAIMGSHMLEVDPTLASDKPRVEVHPLDIGDKADPARLVFTGSEGEGIDVTLSYFDDGYKFIAYDVDCAKPEKEMPNLPVAKQMWTPKCGLKKGATAWMHNGGGHHTVLSMNLTMDQMATLANLFGIELIDIK

SEQ ID NO: 5 - DNA, 1446 bp, Artificial Sequence
  modified L-arabinose isomerase, N-terminal 6xhis-tag, codon optimized
atgcatcaccaccatcatcacgagaacttatattttcaaagcttgaagaccccagattacgaattctggttcgctgtcggttcccaaccattgtacggtccagaagctttggaacaagtcgaaaaggatggtagaaagttggtcgatggtttgaacaagaaggacttgccatacaaggtcgttttcaagttggtcgctaccactgctgattccatcaccaagttcatgaaggatgctaactacaacgataaggtcgctggtgtcatgacctggatgcataccttctccccagctaagaactggatcagaggtacccaattgttacaaaagccattgttacatttggctacccaaatgttggatcatatcccattcgataccatcgacttcgattacatgaacttgaaccaatccgctcatggtgatagagaatacgattacatcaacgctagattgggtgtcccagaaaagatcgtctacggttggtggaacgatccagaagtccaagaagaaatcgctgattggcaaaaggtcgctatcgcctacgatatgtccttcaagatcaagattgctagattcggtgataccatgagagatgtcgctgttaccgaaggtgataaggtcgctgcccaaatcaagttgggttggaccgtcgattactggccagtcgctgaattggtcgctgccgtcaacgctgtctccgaagctgaaatcgatgaacaatacaagagattggaagataactacgacattgacccaaaggcttctatggattccgtcagataccaattgagagaatacttgggtatcaagcatttcttggatgaaaagggttacgatgctttcaccactaacttccaagatttggaaggtttggaacaattgccaggtttggctgtccaattgttaatgatcgatggttacggtttcggtgctgaaggtgatttcaagtccgctggtttgtctagattgttaaagatcgctgccgaaaacaaggaaaccgctttcatggaagattacaccttggatttgagagctggtcatcaagctatcatgggttcccatatgttggaagtcgatccaaccttggcttccgataagccaagagtcgaagttcatccattggatatcggtgacaaggctgatccagctagattggtcttcaccggttccgaaggtgaaggtatcgatgtcaccttgtcctacttcgatgacggttacaagttcatcgcttacgatgtcgactgtgctaagccagaaaaggaaatgccaaacttgccagtcgctaagcaaatgtggaccccaaagtgtggtttgaagaaaggtgctaccgcctggatgcataacggtggtggtcatcacaccgtcttgtccatgaacttgaccatggatcaaatggctaccttggctaacttgttcggtatcgaattgattgatatcaagtga

SEQ ID NO: 6 - protein, 481 aa, Artificial Sequence
  modified L-arabinose isomerase, N-terminal 6xhis-tag
MHHHHHHENLYFQSLKTPDYEFWFAVGSQPLYGPEALEQVEKDGRKLVDGLNKKDLPYKVVFKLVATTADSITKFMKDANYNDKVAGVMTWMHTFSPAKNWIRGTQLLQKPLLHLATQMLDHIPFDTIDFDYMNLNQSAHGDREYDYINARLGVPEKIVYGWWNDPEVQEEIADWQKVAIAYDMSFKIKIARFGDTMRDVAVTEGDKVAAQIKLGWTVDYWPVAELVAAVNAVSEAEIDEQYKRLEDNYDIDPKASMDSVRYQLREYLGIKHFLDEKGYDAFTTNFQDLEGLEQLPGLAVQLLMIDGYGFGAEGDFKSAGLSRLLKIAAENKETAFMEDYTLDLRAGHQAIMGSHMLEVDPTLASDKPRVEVHPLDIGDKADPARLVFTGSEGEGIDVTLSYFDDGYKFIAYDVDCAKPEKEMPNLPVAKQMWTPKCGLKKGATAWMHNGGGHHTVLSMNLTMDQMATLANLFGIELIDIK

SEQ ID NO: 7 - DNA, 1464 bp, Artificial Sequence
  wildtype L-arabinose isomerase, N-terminal 6xhis-tag, codon-optimized
atgcatcaccaccatcatcacgagaacttatattttcaaagcttgaagaccccagattacgaattctggttcgctgtcggttcccaaccattgtacggtccagaagctttggaacaagtcgaaaaggatggtagaaagttggtcgatggtttgaacgcttccggtaagttgccatacaaggtcgttttcaagttggtcgctaccactgctgattccatcaccaagttcatgaaggatgctaactacaacgataaggtcgctggtgtcatgacctggatgcataccttctccccagctaagaactggatcagaggtacccaattgttacaaaagccattgttacatttggctacccaaatgttggatcatatcccattcgataccatcgacttcgattacatgaacttgaaccaatccgctcatggtgatagagaatacgattacatcaacgctagattgggtgtcccagaaaagatcgtctacggttggtggaacgatccagaagtccaagaagaaatcgctgattggcaaaaggtcgctatcgcctacgatatgtccttcaagatcaagattgctagattcggtgataccatgagagatgtcgctgttaccgaaggtgataaggtcgctgcccaaatcaagttgggttggaccgtcgattactggccagtcgctgaattggtcgctgccgtcaacgctgtctccgaagctgaaatcgatgaacaatacaagagattggaagataactacgacatggtcgaaggtgataacgaccatgataagtacgtccattccgtcagataccaattgagagaatacttgggtatcaagcatttcttggatgaaaagggttacgatgctttcaccactaacttccaagatttggaaggtttggaacaattgccaggtttggctgtccaattgttaatgatcgatggttacggtttcggtgctgaaggtgatttcaagtccgctggtttgtctagattgttaaagatcgctgccgaaaacaaggaaaccgctttcatggaagattacaccttggatttgagagctggtcatcaagctatcatgggttcccatatgttggaagtcgatccaaccttggcttccgataagccaagagtcgaagttcatccattggatatcggtgacaaggctgatccagctagattggtcttcaccggttccgaaggtgaaggtatcgatgtcaccttgtcctacttcgatgacggttacaagttcatcgcttacgatgtcgactgtgctaagccagaaaaggaaatgccaaacttgccagtcgctaagcaaatgtggaccccaaagtgtggtttgaagaaaggtgctaccgcctggatgcataacggtggtggtcatcacaccgtcttgtccatgaacttgaccatggatcaaatggctaccttggctaacttgttcggtatcgaattgattgatatcaagtga

SEQ ID NO: 8 - protein, 487 aa, Artificial Sequence
  wildtype L-arabinose isomerase, N-terminal 6xhis-tag
MHHHHHHENLYFQSLKTPDYEFWFAVGSQPLYGPEALEQVEKDGRKLVDGLNASGKLPYKVVFKLVATTADSITKFMKDANYNDKVAGVMTWMHTFSPAKNWIRGTQLLQKPLLHLATQMLDHIPFDTIDFDYMNLNQSAHGDREYDYINARLGVPEKIVYGWWNDPEVQEEIADWQKVAIAYDMSFKIKIARFGDTMRDVAVTEGDKVAAQIKLGWTVDYWPVAELVAAVNAVSEAEIDEQYKRLEDNYDMVEGDNDHDKYVHSVRYQLREYLGIKHFLDEKGYDAFTTNFQDLEGLEQLPGLAVQLLMIDGYGFGAEGDFKSAGLSRLLKIAAENKETAFMEDYTLDLRAGHQAIMGSHMLEVDPTLASDKPRVEVHPLDIGDKADPARLVFTGSEGEGIDVTLSYFDDGYKFIAYDVDCAKPEKEMPNLPVAKQMWTPKCGLKKGATAWMHNGGGHHTVLSMNLTMDQMATLANLFGIELIDIK

SEQ ID NO: 9 - DNA, 1407 bp, Artificial Sequence
  modified L-arabinose isomerase, codon optimized
atgttgaagactccagactacgaattctggttcgctgttggttctcaaccattgtacggtccagaagctttggaacaagttgaaaaggacggtagaaagttggttgacggtttgaacaagaaggacttgccatacaaggttgtcttcaagttggttgctactaccgctgactctattactaagttcatgaaggacgctaactacaacgacaaggttgctggtgttatgacttggatgcacactttctctccagctaagaactggattagaggtactcaattgttgcaaaagccattgttgcacttggctactcaaatgttggaccacattccattcgacactattgatttcgactacatgaacttgaaccaatctgctcacggtgacagagaatacgactacattaacgctagattgggtgttccagaaaagattgtttacggttggtggaacgacccagaagttcaagaagaaattgctgactggcaaaaggttgctattgcttacgacatgtctttcaagattaagatcgctagattcggtgacactatgagagacgttgctgtcactgaaggtgacaaggttgctgctcaaattaagttgggttggactgttgactactggccagttgctgaattggttgctgctgttaacgctgtttctgaagctgaaattgacgaacaatacaagagattggaagacaactacgatattgacccaaaggcttctatggactctgttagataccaattgagagaatacttgggtattaagcacttcttggacgaaaagggttacgacgctttcactaccaacttccaagacttggaaggtttggaacaattgccaggtttggctgttcaattgttgatgattgacggttacggtttcggtgctgaaggtgacttcaagtctgctggtttgtctagattgttgaagattgctgctgaaaacaaggaaactgctttcatggaagactacactttggacttgagagctggtcaccaagctattatgggttctcacatgttggaagttgacccaactttggcttctgacaagccaagagttgaagtccacccattggacattggtgataaggctgacccagctagattggttttcactggttctgaaggtgaaggtattgacgttactttgtcttacttcgacgatggttacaagttcattgcttacgacgttgattgtgctaagccagaaaaggaaatgccaaacttgccagttgctaagcaaatgtggactccaaagtgtggtttgaagaagggtgctactgcttggatgcacaacggtggtggtcaccatactgttttgtctatgaacttgactatggaccaaatggctactttggctaacttgttcggtattgaattgatcgacattaagtaa

SEQ ID NO: 10 - DNA, 1410 bp, Artificial Sequence
  modified L-arabinose isomerase, codon-optimized
atgttgaagaccccagattacgaattctggttcgctgtcggttcccaaccattgtacggtccagaagctttggaacaagtcgaaaaggatggtagaaagttggtcgatggtttgaacgcttccggtaagttgccatacaaggtcgttttcaagttggtcgctaccactgctgattccatcaccaagttcatgaaggatgctaactacaacgataaggtcgctggtgtcatgacctggatgcataccttctccccagctaagaactggatcagaggtacccaattgttacaaaagccattgttacatttggctacccaaatgttggatcatatcccattcgataccatcgacttcgattacatgaacttgaaccaatccgctcatggtgatagagaatacgattacatcaacgctagattgggtgtcccagaaaagatcgtctacggttggtggaacgatccagaagtccaagaagaaatcgctgattggcaaaaggtcgctatcgcctacgatatgtccttcaagatcaagattgctagattcggtgataccatgagagatgtcgctgttaccgaaggtgataaggtcgctgcccaaatcaagttgggttggaccgtcgattactggccagtcgctgaattggtcgctgccgtcaacgctgtctccgaagctgaaatcgatgaacaatacaagagattggaagataactacgacattgacccaaaggcttctatggattccgtcagataccaattgagagaatacttgggtatcaagcatttcttggatgaaaagggttacgatgctttcaccactaacttccaagatttggaaggtttggaacaattgccaggtttggctgtccaattgttaatgatcgatggttacggtttcggtgctgaaggtgatttcaagtccgctggtttgtctagattgttaaagatcgctgccgaaaacaaggaaaccgctttcatggaagattacaccttggatttgagagctggtcatcaagctatcatgggttcccatatgttggaagtcgatccaaccttggcttccgataagccaagagtcgaagttcatccattggatatcggtgacaaggctgatccagctagattggtcttcaccggttccgaaggtgaaggtatcgatgtcaccttgtcctacttcgatgacggttacaagttcatcgcttacgatgtcgactgtgctaagccagaaaaggaaatgccaaacttgccagtcgctaagcaaatgtggaccccaaagtgtggtttgaagaaaggtgctaccgcctggatgcataacggtggtggtcatcacaccgtcttgtccatgaacttgaccatggatcaaatggctaccttggctaacttgttcggtatcgaattgattgatatcaagtga

SEQ ID NO: 11 - protein, 469 aa, Artificial Sequence
  modified L-arabinose isomerase
MLKTPDYEFWFAVGSQPLYGPEALEQVEKDGRKLVDGLNASGKLPYKVVFKLVATTADSITKFMKDANYNDKVAGVMTWMHTFSPAKNWIRGTQLLQKPLLHLATQMLDHIPFDTIDFDYMNLNQSAHGDREYDYINARLGVPEKIVYGWWNDPEVQEEIADWQKVAIAYDMSFKIKIARFGDTMRDVAVTEGDKVAAQIKLGWTVDYWPVAELVAAVNAVSEAEIDEQYKRLEDNYDIDPKASMDSVRYQLREYLGIKHFLDEKGYDAFTTNFQDLEGLEQLPGLAVQLLMIDGYGFGAEGDFKSAGLSRLLKIAAENKETAFMEDYTLDLRAGHQAIMGSHMLEVDPTLASDKPRVEVHPLDIGDKADPARLVFTGSEGEGIDVTLSYFDDGYKFIAYDVDCAKPEKEMPNLPVAKQMWTPKCGLKKGATAWMHNGGGHHTVLSMNLTMDQMATLANLFGIELIDIK

SEQ ID NO: 12 - DNA, 1422 bp, Artificial Sequence
  modified L-arabinose isomerase, codon-optimized
atgttgaagaccccagattacgaattctggttcgctgtcggttcccaaccattgtacggtccagaagctttggaacaagtcgaaaaggatggtagaaagttggtcgatggtttgaacaagaaggacttgccatacaaggtcgttttcaagttggtcgctaccactgctgattccatcaccaagttcatgaaggatgctaactacaacgataaggtcgctggtgtcatgacctggatgcataccttctccccagctaagaactggatcagaggtacccaattgttacaaaagccattgttacatttggctacccaaatgttggatcatatcccattcgataccatcgacttcgattacatgaacttgaaccaatccgctcatggtgatagagaatacgattacatcaacgctagattgggtgtcccagaaaagatcgtctacggttggtggaacgatccagaagtccaagaagaaatcgctgattggcaaaaggtcgctatcgcctacgatatgtccttcaagatcaagattgctagattcggtgataccatgagagatgtcgctgttaccgaaggtgataaggtcgctgcccaaatcaagttgggttggaccgtcgattactggccagtcgctgaattggtcgctgccgtcaacgctgtctccgaagctgaaatcgatgaacaatacaagagattggaagataactacgacatggtcgaaggtgataacgaccatgataagtacgtccattccgtcagataccaattgagagaatacttgggtatcaagcatttcttggatgaaaagggttacgatgctttcaccactaacttccaagatttggaaggtttggaacaattgccaggtttggctgtccaattgttaatgatcgatggttacggtttcggtgctgaaggtgatttcaagtccgctggtttgtctagattgttaaagatcgctgccgaaaacaaggaaaccgctttcatggaagattacaccttggatttgagagctggtcatcaagctatcatgggttcccatatgttggaagtcgatccaaccttggcttccgataagccaagagtcgaagttcatccattggatatcggtgacaaggctgatccagctagattggtcttcaccggttccgaaggtgaaggtatcgatgtcaccttgtcctacttcgatgacggttacaagttcatcgcttacgatgtcgactgtgctaagccagaaaaggaaatgccaaacttgccagtcgctaagcaaatgtggaccccaaagtgtggtttgaagaaaggtgctaccgcctggatgcataacggtggtggtcatcacaccgtcttgtccatgaacttgaccatggatcaaatggctaccttggctaacttgttcggtatcgaattgattgatatcaagtga

SEQ ID NO: 13 - protein, 473 aa, Artificial Sequence
  modified L-arabinose isomerase
MLKTPDYEFWFAVGSQPLYGPEALEQVEKDGRKLVDGLNKKDLPYKVVFKLVATTADSITKFMKDANYNDKVAGVMTWMHTFSPAKNWIRGTQLLQKPLLHLATQMLDHIPFDTIDFDYMNLNQSAHGDREYDYINARLGVPEKIVYGWWNDPEVQEEIADWQKVAIAYDMSFKIKIARFGDTMRDVAVTEGDKVAAQIKLGWTVDYWPVAELVAAVNAVSEAEIDEQYKRLEDNYDMVEGDNDHDKYVHSVRYQLREYLGIKHFLDEKGYDAFTTNFQDLEGLEQLPGLAVQLLMIDGYGFGAEGDFKSAGLSRLLKIAAENKETAFMEDYTLDLRAGHQAIMGSHMLEVDPTLASDKPRVEVHPLDIGDKADPARLVFTGSEGEGIDVTLSYFDDGYKFIAYDVDCAKPEKEMPNLPVAKQMWTPKCGLKKGATAWMHNGGGHHTVLSMNLTMDQMATLANLFGIELIDIK

SEQ ID NO: 14 - DNA, 1449 bp, Artificial Sequence
  modified L-arabinose isomerase, N-terminal 6xhis-tag, codon optimized
atgcatcaccaccatcatcacgagaacttatattttcaaagcttgaagaccccagattacgaattctggttcgctgtcggttcccaaccattgtacggtccagaagctttggaacaagtcgaaaaggatggtagaaagttggtcgatggtttgaacgcttccggtaagttgccatacaaggtcgttttcaagttggtcgctaccactgctgattccatcaccaagttcatgaaggatgctaactacaacgataaggtcgctggtgtcatgacctggatgcataccttctccccagctaagaactggatcagaggtacccaattgttacaaaagccattgttacatttggctacccaaatgttggatcatatcccattcgataccatcgacttcgattacatgaacttgaaccaatccgctcatggtgatagagaatacgattacatcaacgctagattgggtgtcccagaaaagatcgtctacggttggtggaacgatccagaagtccaagaagaaatcgctgattggcaaaaggtcgctatcgcctacgatatgtccttcaagatcaagattgctagattcggtgataccatgagagatgtcgctgttaccgaaggtgataaggtcgctgcccaaatcaagttgggttggaccgtcgattactggccagtcgctgaattggtcgctgccgtcaacgctgtctccgaagctgaaatcgatgaacaatacaagagattggaagataactacgacattgacccaaaggcttctatggattccgtcagataccaattgagagaatacttgggtatcaagcatttcttggatgaaaagggttacgatgctttcaccactaacttccaagatttggaaggtttggaacaattgccaggtttggctgtccaattgttaatgatcgatggttacggtttcggtgctgaaggtgatttcaagtccgctggtttgtctagattgttaaagatcgctgccgaaaacaaggaaaccgctttcatggaagattacaccttggatttgagagctggtcatcaagctatcatgggttcccatatgttggaagtcgatccaaccttggcttccgataagccaagagtcgaagttcatccattggatatcggtgacaaggctgatccagctagattggtcttcaccggttccgaaggtgaaggtatcgatgtcaccttgtcctacttcgatgacggttacaagttcatcgcttacgatgtcgactgtgctaagccagaaaaggaaatgccaaacttgccagtcgctaagcaaatgtggaccccaaagtgtggtttgaagaaaggtgctaccgcctggatgcataacggtggtggtcatcacaccgtcttgtccatgaacttgaccatggatcaaatggctaccttggctaacttgttcggtatcgaattgattgatatcaagtga

SEQ ID NO: 15 - protein, 482 aa, Artificial Sequence
  modified L-arabinose isomerase, N-terminal 6xhis-tag
MHHHHHHENLYFQSLKTPDYEFWFAVGSQPLYGPEALEQVEKDGRKLVDGLNASGKLPYKVVFKLVATTADSITKFMKDANYNDKVAGVMTWMHTFSPAKNWIRGTQLLQKPLLHLATQMLDHIPFDTIDFDYMNLNQSAHGDREYDYINARLGVPEKIVYGWWNDPEVQEEIADWQKVAIAYDMSFKIKIARFGDTMRDVAVTEGDKVAAQIKLGWTVDYWPVAELVAAVNAVSEAEIDEQYKRLEDNYDIDPKASMDSVRYQLREYLGIKHFLDEKGYDAFTTNFQDLEGLEQLPGLAVQLLMIDGYGFGAEGDFKSAGLSRLLKIAAENKETAFMEDYTLDLRAGHQAIMGSHMLEVDPTLASDKPRVEVHPLDIGDKADPARLVFTGSEGEGIDVTLSYFDDGYKFIAYDVDCAKPEKEMPNLPVAKQMWTPKCGLKKGATAWMHNGGGHHTVLSMNLTMDQMATLANLFGIELIDIK

SEQ ID NO: 16 - DNA, 1461 bp, Artificial Sequence
  modified L-arabinose isomerase, N-terminal 6xhis-tag, codon optimized
atgcatcaccaccatcatcacgagaacttatattttcaaagcttgaagaccccagattacgaattctggttcgctgtcggttcccaaccattgtacggtccagaagctttggaacaagtcgaaaaggatggtagaaagttggtcgatggtttgaacaagaaggacttgccatacaaggtcgttttcaagttggtcgctaccactgctgattccatcaccaagttcatgaaggatgctaactacaacgataaggtcgctggtgtcatgacctggatgcataccttctccccagctaagaactggatcagaggtacccaattgttacaaaagccattgttacatttggctacccaaatgttggatcatatcccattcgataccatcgacttcgattacatgaacttgaaccaatccgctcatggtgatagagaatacgattacatcaacgctagattgggtgtcccagaaaagatcgtctacggttggtggaacgatccagaagtccaagaagaaatcgctgattggcaaaaggtcgctatcgcctacgatatgtccttcaagatcaagattgctagattcggtgataccatgagagatgtcgctgttaccgaaggtgataaggtcgctgcccaaatcaagttgggttggaccgtcgattactggccagtcgctgaattggtcgctgccgtcaacgctgtctccgaagctgaaatcgatgaacaatacaagagattggaagataactacgacatggtcgaaggtgataacgaccatgataagtacgtccattccgtcagataccaattgagagaatacttgggtatcaagcatttcttggatgaaaagggttacgatgctttcaccactaacttccaagatttggaaggtttggaacaattgccaggtttggctgtccaattgttaatgatcgatggttacggtttcggtgctgaaggtgatttcaagtccgctggtttgtctagattgttaaagatcgctgccgaaaacaaggaaaccgctttcatggaagattacaccttggatttgagagctggtcatcaagctatcatgggttcccatatgttggaagtcgatccaaccttggcttccgataagccaagagtcgaagttcatccattggatatcggtgacaaggctgatccagctagattggtcttcaccggttccgaaggtgaaggtatcgatgtcaccttgtcctacttcgatgacggttacaagttcatcgcttacgatgtcgactgtgctaagccagaaaaggaaatgccaaacttgccagtcgctaagcaaatgtggaccccaaagtgtggtttgaagaaaggtgctaccgcctggatgcataacggtggtggtcatcacaccgtcttgtccatgaacttgaccatggatcaaatggctaccttggctaacttgttcggtatcgaattgattgatatcaagtga

SEQ ID NO: 17 - protein, 486 aa, Artificial Sequence
  modified L-arabinose isomerase, N-terminal 6xhis-tag
MHHHHHHENLYFQSLKTPDYEFWFAVGSQPLYGPEALEQVEKDGRKLVDGLNKKDLPYKVVFKLVATTADSITKFMKDANYNDKVAGVMTWMHTFSPAKNWIRGTQLLQKPLLHLATQMLDHIPFDTIDFDYMNLNQSAHGDREYDYINARLGVPEKIVYGWWNDPEVQEEIADWQKVAIAYDMSFKIKIARFGDTMRDVAVTEGDKVAAQIKLGWTVDYWPVAELVAAVNAVSEAEIDEQYKRLEDNYDMVEGDNDHDKYVHSVRYQLREYLGIKHFLDEKGYDAFTTNFQDLEGLEQLPGLAVQLLMIDGYGFGAEGDFKSAGLSRLLKIAAENKETAFMEDYTLDLRAGHQAIMGSHMLEVDPTLASDKPRVEVHPLDIGDKADPARLVFTGSEGEGIDVTLSYFDDGYKFIAYDVDCAKPEKEMPNLPVAKQMWTPKCGLKKGATAWMHNGGGHHTVLSMNLTMDQMATLANLFGIELIDIK